(19) United States
(12) Patent Application Publication
(10) Pub. No.: US 2026/0338318 A1
(43) Pub. Date: Oct. 29, 2026

(54) ADJUSTABLE ARM ASSEMBLY

(71) Applicant: Medtronic Navigation, Inc., Lafayette, CO (US)

(72) Inventors: Sanjay YADAV, Indore (IN); Nilesh Anant JANGALI, Nallagandla Hyderabad (IN)

(21) Appl. No.: 19/191,657

(22) Filed: Apr. 28, 2025

(51) Int. Cl. F16M 13/02; F16M 11/12; F16M 11/20

(52) U.S. Cl. CPC F16M 13/02; F16M 11/125; F16M 11/2064

(57) ABSTRACT

In some aspects, an adjustable arm assembly may include a base mount, a first segment, a second segment, and a display mount. The first segment may be affixed to the base mount and configured to rotate about a first vertical axis. The second segment may be affixed to the first segment and configured to rotate about a second vertical axis and a first lateral axis. The display mount may be affixed to the second segment and configured to rotate about a third vertical axis, a second lateral axis, and a longitudinal axis.

### BACKGROUND

Medical imaging data includes various imaging techniques that can be used to visualize internal body structures for diagnostic and therapeutic purposes. These images are crucial for assessing health conditions, guiding treatment decisions, and/or monitoring disease progression, among other examples. During a procedure (e.g., surgery), real-time imaging can assist surgeons by providing information associated with the anatomy of a patient, allowing for more precise interventions and improved patient outcomes.

### SUMMARY

Some implementations described herein relate to an adjustable arm assembly for a medical imaging system. The adjustable arm assembly may include a base mount. The adjustable arm assembly may include a first segment affixed to the base mount and configured to rotate about a first vertical axis. The adjustable arm assembly may include a second segment affixed to the first segment and configured to rotate about a second vertical axis and a first lateral axis. The adjustable arm assembly may include a display mount affixed to the second segment and configured to rotate about a third vertical axis, a second lateral axis, and a longitudinal axis.

Some implementations described herein relate to a medical imaging system. The medical imaging system may include a medical imaging device and an adjustable arm assembly. The adjustable arm assembly may include a base mount affixed to the medical imaging device. The adjustable arm assembly may include a first segment affixed to the base mount and configured to rotate about a first vertical axis. The adjustable arm assembly may include a second segment affixed to the first segment and configured to rotate about a second vertical axis and a first lateral axis. The adjustable arm assembly may include a display mount affixed to the second segment and configured to rotate about a third vertical axis, a second lateral axis, and a longitudinal axis.

Some implementations described herein relate to an adjustable arm assembly for a medical imaging system. The adjustable arm assembly may include a base mount. The adjustable arm assembly may include a first segment affixed to the base mount and configured to rotate about a first vertical axis. The adjustable arm assembly may include a second segment affixed to the first segment and configured to rotate 360 degrees about a second vertical axis and at least 120 degrees about a first lateral axis. The adjustable arm assembly may include a display mount affixed to the second segment and configured to rotate about a third vertical axis, at least 180 degrees about a second lateral axis, and about a longitudinal axis.

### BRIEF DESCRIPTION OF THE DRAWINGS

FIG. 1 is a diagram of a perspective view of an adjustable arm assembly.

FIG. 2 is a diagram of an example implementation associated with a rotatability of the adjustable arm assembly.

FIG. 3 is a diagram of a back view of the adjustable arm assembly.

FIG. 4 is a diagram of a bottom view of the adjustable arm assembly.

FIG. 5 is a diagram of a side view of the adjustable arm assembly.

FIGS. 6A-6C are diagrams of example implementations associated with a collapsed state of the adjustable arm assembly.

FIG. 7 is an example implementation associated with an expanded state of the adjustable arm assembly.

FIG. 8 is a diagram of an example medical imaging system.

### DETAILED DESCRIPTION

The following detailed description of example aspects refers to the accompanying drawings. The same reference numbers in different drawings may identify the same or similar elements.

Medical imaging devices may include user input devices that allow users (e.g., surgeons) to control medical imaging parameters and/or perform medical imaging operations. For example, a user may, via a user input device, input data (e.g., X-ray parameters), control an X-ray collimator shutter and/or gantry motion, save acquired medical images, or the like. The user input device may include a screen (e.g., a liquid crystal display (LCD) screen) and/or one or more dedicated keys that enable the user to control the medical imaging parameters and/or perform the medical imaging operations.

In some examples, the user input device may have a fixed placement and orientation relative to the medical imaging device. For example, the user input device may be fixed at a backside of a gantry of the medical imaging device. As a result, the user input device can be difficult to operate, such as during a medical imaging procedure. For example, the user may be able to engage with the user input device only while standing near the backside of the gantry. Additionally, or alternatively, the user input device may be incapable of displaying a medical image acquired by the medical imaging device; as a result, the user may view the medical image on a separate mobile view station, which can occupy significant space in an operation theater (OT).

Some aspects described herein relate to an adjustable arm that attaches a display device to a medical imaging device. In some examples, the adjustable arm and the display device may replace the user input device described above. The adjustable arm may include a base mount that connects to the medical imaging device, a display mount that connects to the display device, and two segments between the base mount and the display mount. A first segment may connect to the base mount and rotate about a vertical axis. A second segment may connect to the first segment and rotate about vertical and lateral axes. The display mount may connect to the second segment and rotate about vertical, lateral, and longitudinal axes. The adjustable arm, which may be referred to as a “flexible arm” or “flex arm,” may be constructed from any suitable material(s), such as plastic, metal, and/or the like. In some examples, the display device may be mounted on the display mount. For example, the display device may be a touch monitor providing a user interface (UI) that enables the user to control medical imaging parameters and/or perform medical imaging operations. For example, the UI may provide any suitable quantity of interface keys that are selectable via touch. For example, the UI may show X-ray collimator shutter and/or gantry motion and enable control of the same. Additionally, or alternatively, the display device may display one or more medical images acquired by the medical imaging device (e.g., using X-ray imaging or the like).

As a result, the adjustable arm may enable the user to move the display device to various placements and/or orientations, such as during medical imaging procedures. Thus, the user may control medical imaging parameters and/or perform medical imaging operations from any suitable location (rather than only near the backside of the gantry). Additionally, or alternatively, the display device connected to the adjustable arm may enable display of acquired medical images, mitigating a need for a separate mobile view station and, thus, increasing available space in the OT.

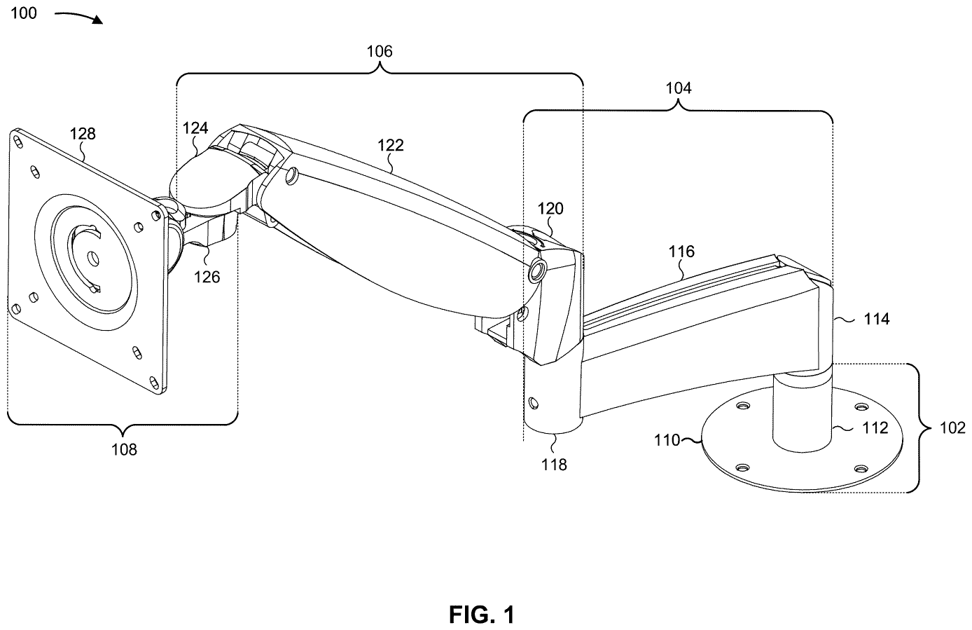

FIG. 1 is a diagram of a perspective view 100 of an adjustable arm assembly.

As shown, the adjustable arm assembly includes a base mount 102, a first segment 104, a second segment 106, and a display mount 108. The base mount 102 includes a circular portion 110 and a cylindrical portion 112. In some aspects, the base mount 102 may be affixed to a medical imaging device. For example, the circular portion 110 of the base mount 102 may be affixed to the medical imaging device (e.g., via a locking mechanism, a screw mechanism, a snap mechanism, or the like). The cylindrical portion 112 may connect to the first segment 104.

The first segment 104 includes a connector portion 114, a shaft portion 116, and a cylindrical portion 118. In some aspects, the first segment 104 may be affixed to the base mount 102. For example, the connector portion 114 of the first segment 104 may be affixed to the cylindrical portion 112 of the base mount 102 (e.g., via a locking mechanism, a screw mechanism, a snap mechanism, or the like). The shaft portion 116 may extend outward from the base mount 102. The cylindrical portion 118 may connect to the second segment 106.

The second segment 106 includes a connector portion 120, a shaft portion 122, and a connector portion 124. In some aspects, the second segment 106 may be affixed to the first segment 104. For example, the connector portion 120 of the second segment 106 may be affixed to the cylindrical portion 118 of the first segment 104 (e.g., via a locking mechanism, a screw mechanism, a snap mechanism, or the like). The shaft portion 122 may extend outward from the cylindrical portion 118. The connector portion 124 may connect to the display mount 108.

The display mount 108 includes a connector portion 126 and a panel 128. In some aspects, the display mount 108 may be affixed to the second segment 106. For example, the connector portion 126 of the display mount 108 may be affixed to the connector portion 124 of the second segment 106 (e.g., via a locking mechanism, a screw mechanism, a snap mechanism, or the like). In some aspects, the display mount may be affixed to a display device. For example, the panel 128 of display mount 108 may be affixed to the display device (e.g., via a locking mechanism, a screw mechanism, a snap mechanism, or the like).

As indicated above, FIG. 1 is provided as an example. Other examples may differ from what is described with regard to FIG. 1.

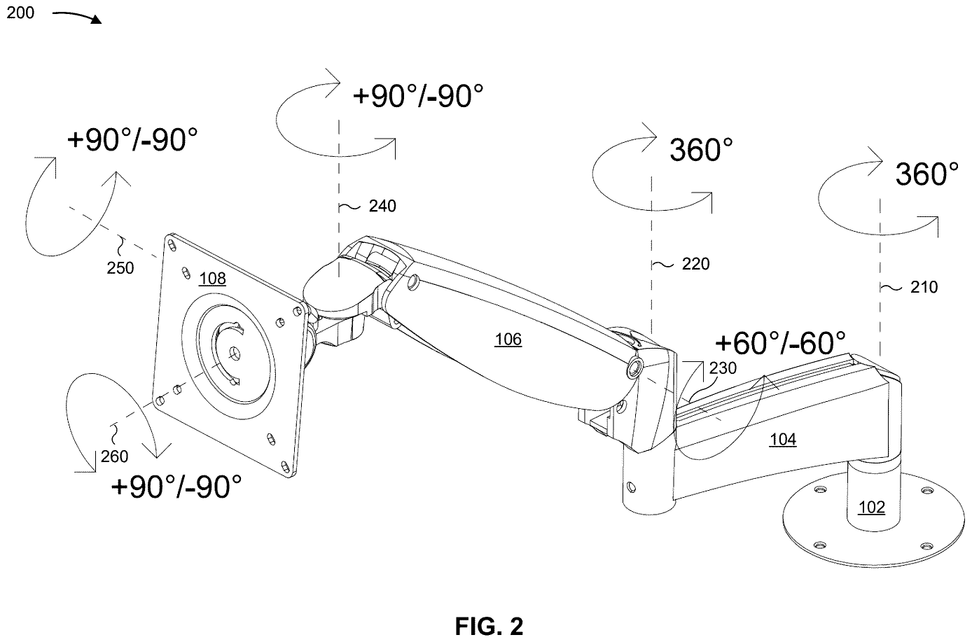

FIG. 2 is a diagram of an example implementation 200 associated with a rotatability of the adjustable arm assembly.

In some aspects, the first segment 104 may be configured to rotate about a first vertical axis 210. For example, the connector portion 114 of the first segment 104 may be configured to rotate relative to the cylindrical portion 112 of the base mount 102 (e.g., via a bearing mechanism, frictional mechanism, ratcheting mechanism, or the like). In some aspects, the first segment 104 may be configured to rotate 360 degrees about the first vertical axis 210.

In some aspects, the second segment 106 may be configured to rotate about a second vertical axis 220. For example, the connector portion 120 of the second segment 106 may be configured to rotate relative to the cylindrical portion 118 of the first segment 104 (e.g., via a bearing mechanism, frictional mechanism, ratcheting mechanism, or the like). In some aspects, the second segment 106 may be configured to rotate 360 degrees about the second vertical axis 220.

In some aspects, the second segment 106 may be configured to rotate about a first lateral axis 230. For example, the shaft portion 122 of the second segment 106 may be configured to rotate relative to the connector portion 120 (e.g., via a bearing mechanism, frictional mechanism, ratcheting mechanism, or the like). In some aspects, the second segment 106 may be configured to rotate at least 120 degrees about the first lateral axis 230.

In some aspects, the display mount 108 may be configured to rotate about a third vertical axis 240. For example, the connector portion 126 of the display mount 108 may be configured to rotate relative to the connector portion 124 of the second segment 106 (e.g., via a bearing mechanism, frictional mechanism, ratcheting mechanism, or the like). In some aspects, the display mount 108 may be configured to rotate at least 180 degrees about the third vertical axis 240.

In some aspects, the display mount 108 may be configured to rotate about a second lateral axis 250. For example, the connector portion 124 of the second segment 106 may be configured to rotate relative to the shaft portion 122 of the second segment 106 (e.g., via a bearing mechanism, frictional mechanism, ratcheting mechanism, or the like). In some aspects, the display mount 108 may be configured to rotate at least 180 degrees about the second lateral axis 250.

In some aspects, the display mount 108 may be configured to rotate about a longitudinal axis 260. For example, the panel 128 of display mount 108 may be configured to rotate relative to the connector portion 126 of the display mount 108 (e.g., via a bearing mechanism, frictional mechanism, ratcheting mechanism, or the like). In some aspects, the display mount 108 may be configured to rotate at least 180 degrees about the longitudinal axis 260.

As indicated above, FIG. 2 is provided as an example. Other examples may differ from what is described with regard to FIG. 2.

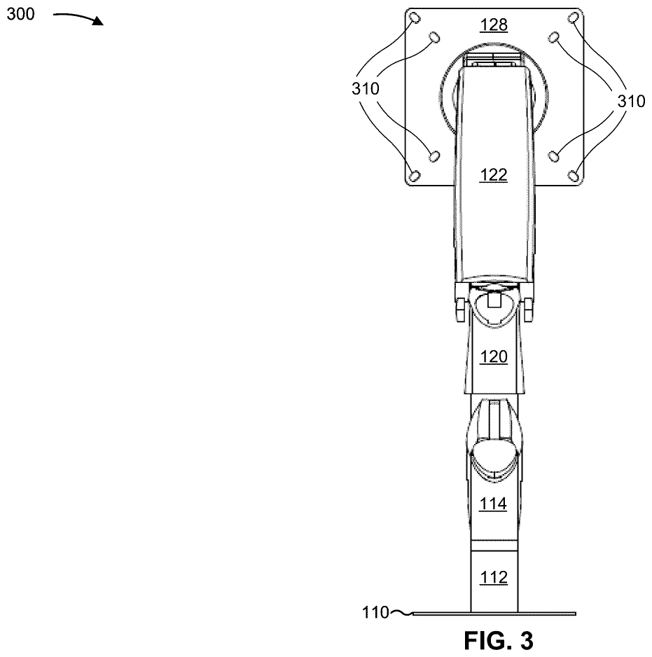

FIG. 3 is a diagram of a back view 300 of the adjustable arm assembly.

As shown, the panel 128 may define a plurality of holes 310 (e.g., through holes). The holes 310 may be arranged in concentric rectangles or squares, where each hole 310 is placed at a corner of a rectangle or square. For example, the panel 128 may define eight holes arranged in two concentric rectangles or squares. In some examples, the holes 310 may be configured to accept respective screws that, when screwed into a display device, help to affix the display device to the display mount 108. For example, the display device may have corresponding holes (e.g., blind holes) that align with holes 310 and are configured to accept the respective screws.

As indicated above, FIG. 3 is provided as an example. Other examples may differ from what is described with regard to FIG. 3.

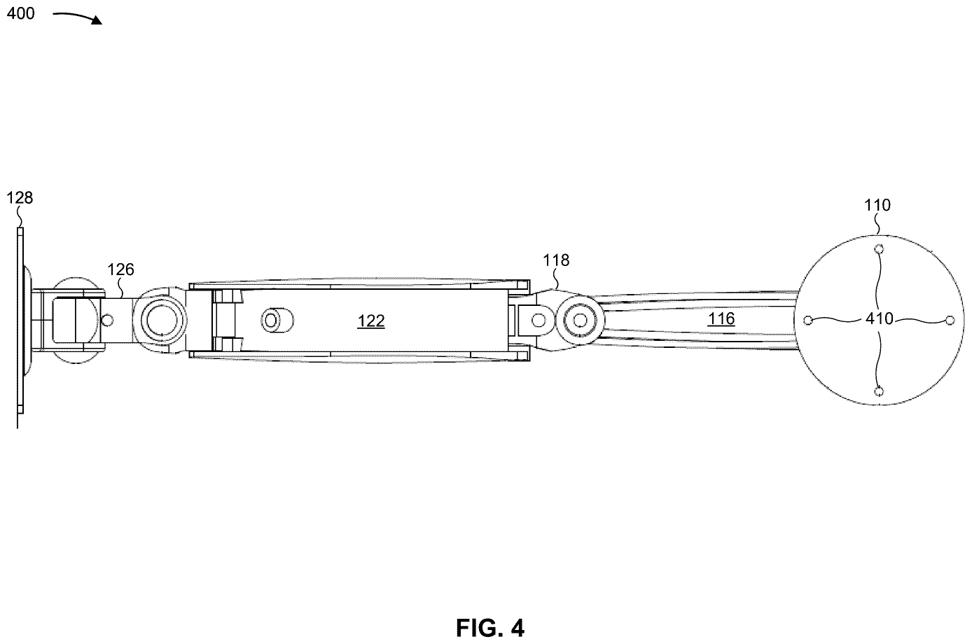

FIG. 4 is a diagram of a bottom view 400 of the adjustable arm assembly.

As shown, the circular portion 110 may define a plurality of holes 410 (e.g., through holes). The holes 410 may be arranged in a circle. For example, the circular portion 110 may define four holes 410 arranged in the circle. In some examples, the holes 410 may be configured to accept respective screws that, when screwed into a medical imaging device, help to affix the adjustable arm assembly to the medical imaging device. For example, the medical imaging device may have corresponding holes (e.g., blind holes) that align with holes 410 and are configured to accept the respective screws.

As indicated above, FIG. 4 is provided as an example. Other examples may differ from what is described with regard to FIG. 4.

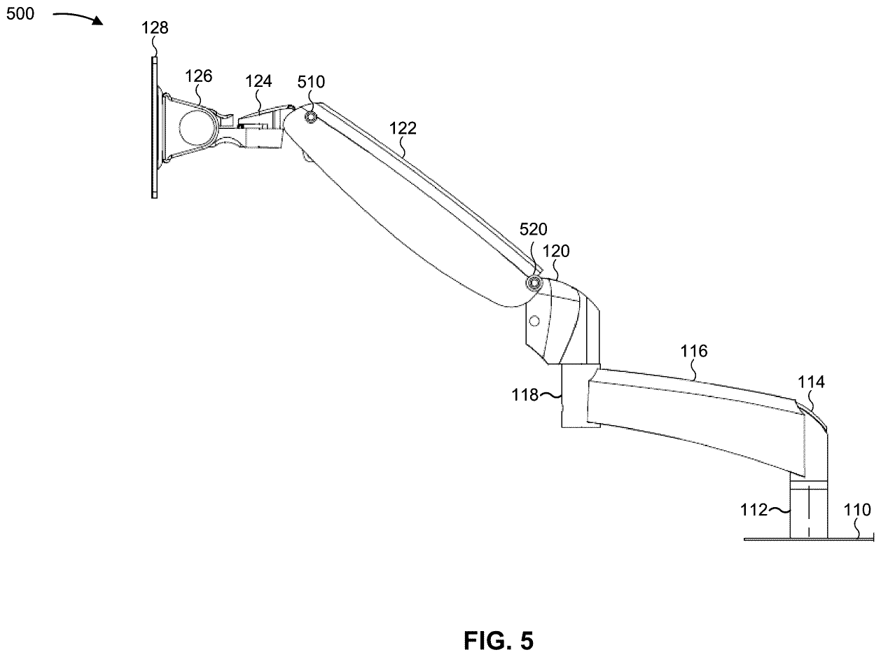

FIG. 5 is a diagram of a side view 500 of the adjustable arm assembly.

As shown, the shaft portion 122 may define a hole 510 and a hole 520. The holes 510 and 520 may be through holes. In some examples, the hole 510 may help to rotatably affix the connector portion 124 to the shaft portion 122. For example, the connector portion 124 may be snapped, screwed, or otherwise affixed to the shaft portion 122 via the hole 510. In some examples, the hole 520 may help to rotatably affix the connector portion 120 to the shaft portion 122. For example, the connector portion 120 may be snapped, screwed, or otherwise affixed to the shaft portion 122 via the hole 520.

As indicated above, FIG. 5 is provided as an example. Other examples may differ from what is described with regard to FIG. 5.

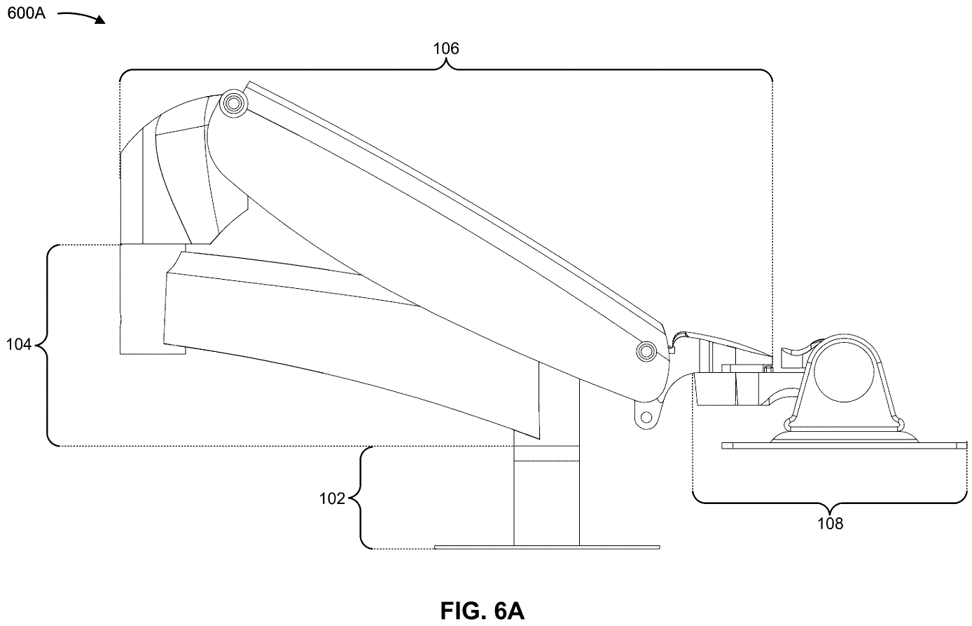

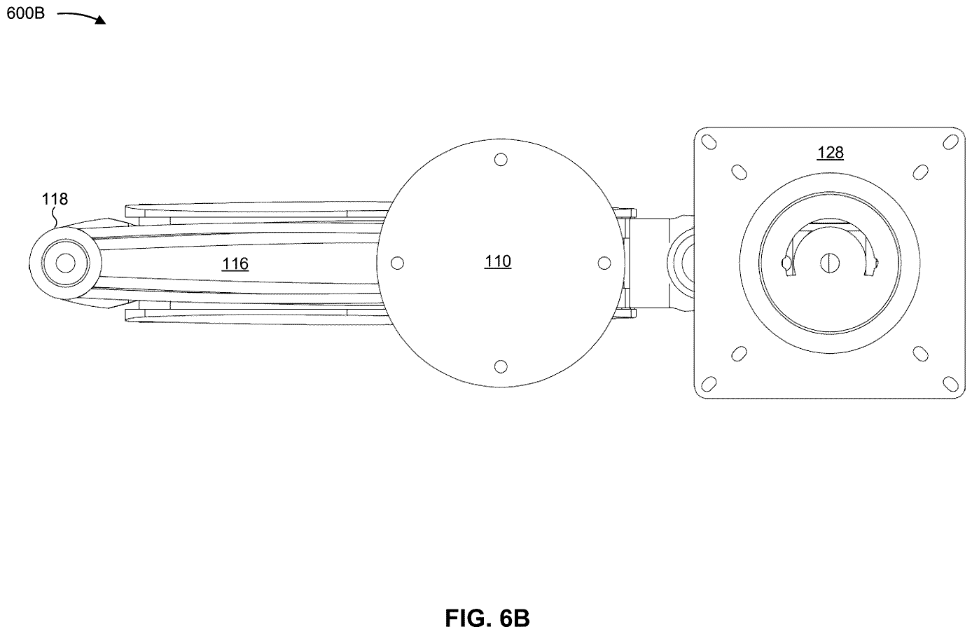

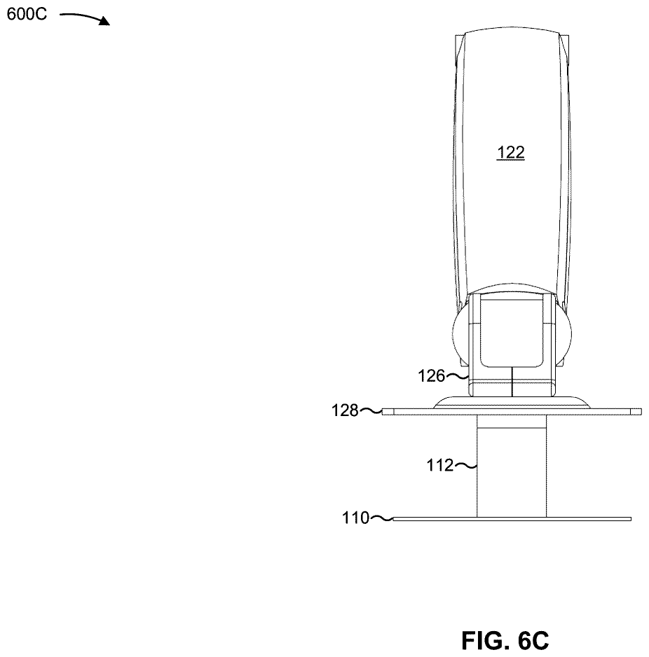

FIGS. 6A-6C are diagrams of example implementations 600A-600C associated with a collapsed state of the adjustable arm assembly.

In some aspects, as discussed above in connection with FIG. 2, the second segment 106 may be configured to rotate 360 degrees about the second vertical axis 220 and at least 120 degrees about the first lateral axis 230, and the display mount 108 may be configured to rotate at least 180 degrees about the second lateral axis 250. This rotatability of the second segment 106 and the display mount 108 may enable the adjustable arm assembly to enter the collapsed state.

With reference to FIG. 6A, shown is a side view of the adjustable arm assembly in the collapsed state. In some examples, a height of the adjustable arm assembly in the collapsed state (e.g., a length from a bottommost part of the base mount 102 to an uppermost part of the second segment 106) may be approximately 250 millimeters. With reference to FIG. 6B, shown is a bottom view of the adjustable arm assembly in the collapsed state. In some examples, a depth of the adjustable arm assembly in the collapsed state (e.g., a length from a leftmost part of the cylindrical portion 118 to a rightmost part of the panel 128) may be approximately 452 millimeters. With reference to FIG. 6C, shown is a bottom view of the adjustable arm assembly in the collapsed state. In some examples, a width of the adjustable arm assembly in the collapsed state (e.g., a length of the panel 128) may be approximately 131 millimeters. Thus, in some examples, the dimensions of the adjustable arm assembly in the collapsed state may be approximately 250 millimeters×452 millimeters×131 millimeters; however, the adjustable arm assembly may have any suitable dimensions in the collapsed state.

As indicated above, FIGS. 6A-6C are provided as examples. Other examples may differ from what is described with regard to FIGS. 6A-6C.

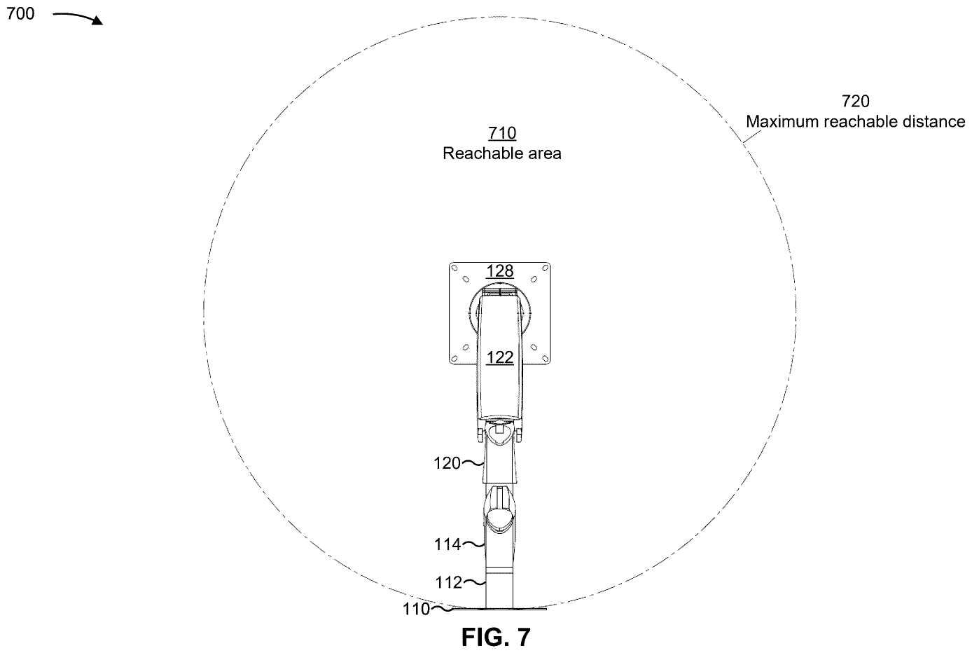

FIG. 7 is an example implementation 700 associated with an expanded state of the adjustable arm assembly.

The adjustable arm assembly may extend within a reachable area 710. For example, the reachable area 710 may be a set of distances between a center of the panel 128 to a center of the circular portion 110. In the expanded state, the adjustable arm assembly may extend to a maximum reachable distance 720, which may be an outermost edge of the reachable area 710. In some examples, the maximum reachable distance 720 may be approximately 656 millimeters; however, the adjustable arm assembly may have any suitable maximum reach in the expanded state.

As indicated above, FIG. 7 is provided as an example. Other examples may differ from what is described with regard to FIG. 7.

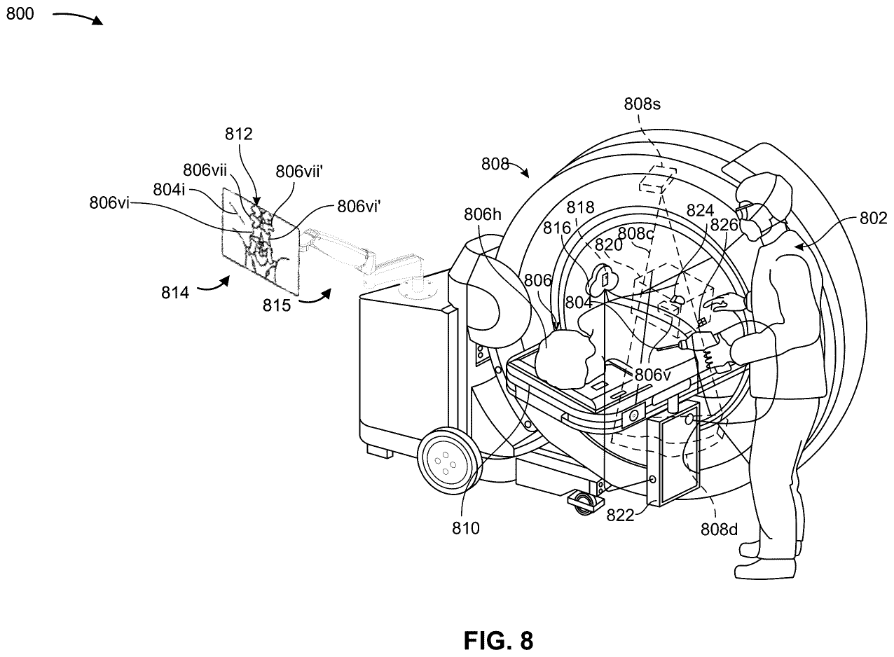

FIG. 8 is a diagram of an example medical imaging system 800. The medical imaging system 800 is depicted in an example procedure room or arena. The medical imaging system 800 may be used for various purposes or procedures by one or more users, such as a user 802. The user 802 may be a surgeon, clinician, nurse, and/or other user.

In some examples, the medical imaging system 800 may be used to determine or track a position of an instrument 804 in a volume. The position may include a three-dimensional location and/or an orientation. The orientation may include one or more degrees of freedom, such as three degrees of freedom, among other examples. Additionally, or alternatively, the medical imaging system 800 may be used to determine or track a position of a patient 806 (e.g., a human subject) and/or one or more anatomical elements of the patient 806.

The instrument 804 may be an implement or tool used as part of a procedure or surgery. For example, the instrument 804 may include an implement, a surgical instrument, a drill, a lead, a cutting tool, a grasper, scissors, an electrocautery tool, a suction tool, a probe (e.g., an optical coherence tomography (OCT) probe, a sonography probe (e.g., an ultrasound transducer), an endocavitary probe, or another type of probe), an imaging device, a Doppler device, an electrocardiogram device, a spectrometer, an X-ray device, a handheld magnetic resonance imaging (MRI) device, a retractor, a stapler, a dissector (e.g., an ultrasonic dissector), a camera (e.g., a thermal imaging camera or an infrared thermography device), an endoscope, a drill, a cutter, a laser, an aspirator (e.g., an ultrasonic aspirator), a trocar, and/or a cannula, among other examples. In some examples, the instrument 804 may include one or more markers to enable tracking by the medical imaging system 800. In some examples, the instrument 804 may include one or more components (e.g., a radio-frequency identification (RFID) component or another component) configured to transmit signal(s) to one or more components of the medical imaging system 800 to enable tracking by the medical imaging system 800.

Tracking the position of the instrument 804 may assist the user 802 in determining a position of the instrument 804, even if the instrument 804 is not directly viewable by the user 802. Various procedures may block the view of the user 802, such as performing a repair or assembling an inanimate system (such as a robotic system), assembling portions of an airframe and/or an automobile, or the like. Various other procedures may include a surgical procedure, such as performing a spinal procedure, neurological procedure, positioning a deep brain simulation probe, and/or other surgical procedures on a living subject (such as the patient 806), among other examples.

The medical imaging system 800 may include a medical imaging device 808 that is operable to image the patient 806. A subject support 810 may be used to support or hold the patient 806 during imaging and/or during a procedure. The subject support 810 may be an operating table. The same or different supports may be used for different portions of a procedure.

In some examples, the image data may be generated by, captured by, and/or provided by (e.g., to an image database) the medical imaging device 808. The medical imaging device 808 may be operable to image anatomical element(s) (e.g., a bone, veins, tissue, and/or other anatomical elements) and/or other aspects of patient anatomy to output medical imaging data (e.g., image data depicting or corresponding to a bone, veins, and/or tissue). “Image data” or “imaging data” as used herein refers to the data generated or captured by an imaging device, including in a machine-readable form, a graphical/visual form, and/or in any other form. In some examples, medical image data may include data corresponding to an anatomical feature of the patient 806, or to a portion thereof. The image data may be or include a preoperative image, an intraoperative image, a postoperative image, or an image taken independently of any procedure. In some examples, a first imaging device may be used to obtain first image data (e.g., a first image, a first video containing a plurality of images, or the like) at a first time, and a second imaging device may be used to obtain second image data (e.g., a second image, a second video containing a plurality of images, or the like) at a second time after the first time. The medical imaging device 808 may be capable of taking a two-dimensional (2D) image or a three-dimensional (3D) image to yield the image data. The medical imaging device 808 is depicted in FIG. 8 as an O-arm imaging device as an example. Additionally, or alternatively, the medical imaging device 808 may be, or may include, an ultrasound scanner (e.g., which may include a physically separate transducer and receiver, or a single ultrasound transceiver), a C-arm imaging system, a G-arm imaging system, or any other device utilizing X-ray-based imaging (e.g., a fluoroscope, a computed tomography (CT) scanner, or other X-ray machine), an MRI scanner, an OCT scanner, an endoscope, a microscope, an optical camera, a thermographic camera (e.g., an infrared camera), a radar system (which may include a transmitter, a receiver, a processor, and one or more antennas), or any other imaging device suitable for obtaining images of an anatomical feature of the patient 806, of the instrument 804, of one or more users 802, and/or other devices, objects, structures, and/or people associated with the medical imaging system 800. In some examples, image data, as described herein, may be considered to be continuous and/or provided as an image data stream, such as if the image data represents two or more frames per second.

In some examples, the medical imaging device 808 may include a source 808s configured to emit and/or generate X-rays. The X-rays may form a cone 808c, such as in a cone beam, that impinge on the patient 806. Some of the X-rays pass though and some are attenuated by the patient 806. The medical imaging device 808 may further include a detector 808d to detect the X-rays that are not completely attenuated, or blocked, by the patient 806. Thus, acquired image data may include X-ray image data. In some examples, the image data may be 2D image data and/or 3D image data. The image data may be acquired during or before a surgical procedure.

The image data may be used to display an image 812 on a display device 814. In some examples, the image data may be used to form or reconstruct selected types of image data, such as three-dimensional volumes. The display device 814 may include a communication device and/or a computing device. The display device 814 may be any device capable of, or configured to, provide, display, or otherwise output (e.g., via a visual output and/or an audio output) information. For example, the display device 814 may include a monitor, such as a screen (e.g., a touchscreen). In some examples, the monitor may be a 10-inch monitor, powered using a 12-volt power supply adapter, and/or connected to a computer wirelessly and/or via a wired connection (e.g., via a high-definition multimedia interface (HDMI), a universal serial bus (USB) interface, and/or the like). Additionally, or alternatively, the display device 814 may include a wireless communication device, a mobile phone, a user equipment, a laptop computer, a tablet computer, a desktop computer, a keyboard, mouse, trackball, television, a speaker, and/or any other device for receiving information from a user and/or for providing information to the user 802.

In some examples, the display device 814 may be connected to the medical imaging device 808 via an adjustable arm assembly 815 as described elsewhere herein. In some examples, the user 802 may use the adjustable arm assembly 815 to position and/or orient the display device 814, as discussed above in connection with FIGS. 1-5. In some examples, the user 802 may configure the adjustable arm assembly 815 in a collapsed state, as discussed above in connection with FIGS. 6A-6C. In some examples, the user 802 may extend the display device to the maximum reachable distance 720, as discussed above in connection with FIG. 7.

The instrument 804, the patient 806 (e.g., one or more anatomical elements of the patient 806), the user 802, and/or other components or objects (e.g., a robot and/or robotic arm) may be tracked in a trackable volume or a navigational volume by one or more tracking systems. Tracking systems may include one or more tracking systems that operate in an identical manner and/or different manners or modes. For example, the tracking system may include an electro-magnetic (EM) localizer 816 and/or one or more other components, such as an optical component, a radar component, and/or an ultrasonic component, among other examples. For example, the position of the instrument 804 may be tracked in the tracking volume relative to the patient 806 and then illustrated as a graphical representation or icon 804i (hereinafter “icon 804i”) at the display device 814. For example, the icon 804i may be superimposed on the image 812 and/or adjacent to the image 812. The medical imaging system 800 may incorporate the display device 814 and operate to render the image 812 from selected image data, display the image 812, determine the position of the instrument 804, and/or determine the position of the icon 804i, among other examples.

The EM localizer 816 may be configured to generate electro-magnetic fields with a transmitting coil array (TCA) 818 that is incorporated into the EM localizer 816. The TCA 818 may include one or more coil groupings or arrays. In some examples, the TCA 818 may include multiple coil groupings or arrays, each including three coils (which may be referred to as “trios” or “triplets”). The coils may be powered to generate or form an electro-magnetic field by driving current through the coils of the coil groupings or arrays. As the current is driven through the coils, the electro-magnetic fields generated may extend away from the coils and form a navigation domain or volume 820. For example, the navigation domain or volume 820 may encompass all or a portion of a head 806h, spinal vertebrae 806v, and/or other appropriate portion of the patient 806. The coils may be powered through a TCA controller and/or power supply 822. In some examples, multiple EM localizers 816 may be provided and each may be placed at different and selected locations.

The navigation domain or volume 820 generally defines a navigation space or patient space. The instrument 804 may be tracked in the navigation space or patient space that is defined by the navigation domain or volume 820 relative to the patient 806 using a tracking device 826. For example, the instrument 804 may be freely moveable (such as by the user 802) relative to a dynamic reference frame (DRF) or patient reference frame tracker 824 that is fixed relative to the subject 806. The tracking device 826 and/or the DRF or patient reference frame tracker 824 may include tracking portions that are tracking with appropriate tracking systems, such as sensing coils (e.g., conductive material formed or placed in a coil) that sense and are used to measure a magnetic field strength, optical reflectors, and/or ultrasonic emitters, among other examples. The tracking device 826 may be connected or associated with the instrument 804 relative to the DRF or patient reference frame tracker 824. The medical imaging system 800 may be used to determine the position of the instrument 804 relative to the DRF or patient reference frame tracker 824. In some examples, the navigation domain or volume 820 may be registered to an image space defined by the image 812 of the patient 806, and the icon 804i representing the instrument 804 may be illustrated at a navigated (e.g., determined) and tracked position using the display device 814.

In some examples, the medical imaging device 808 can generate and/or emit X-rays from the X-ray source that propagate through the patient 806 and are received by the X-ray imaging receiving portion. The image capturing portion generates image data representing the intensities of the received X-rays. Typically, the image capturing portion can include an image intensifier that first converts the X-rays to visible light and a camera (e.g. a charge couple device) that converts the visible light into digital image data. The image capturing portion may also be a digital device that converts X-rays directly to digital image data for forming images, thus potentially avoiding distortion introduced by first converting to visible light.

The image 812 that is displayed with the display device 814 may be based on image data of the patient 806. In some examples, the image data that is used to generate the image 812 may be acquired using the medical imaging device 808 and/or any other suitable (e.g., selected) imaging system (e.g., a magnetic resonance imager, and/or a CT imager). The acquired image data may be 2D image data or 3D image data. In some example, the image data may include a time-varying component, such as image data acquired during a heart rhythm and/or breathing cycle of the patient 806.

In some examples, the image data may be a 2D image data that is generated using a cone beam associated with the cone 808 c. The 2D image data may then be used to reconstruct a 3D image or model of the imaged subject, such as the patient 806. The reconstructed 3D image and/or an image based on the 2D image data may be displayed (e.g., the image 812 may be generated using selected image data).

In some examples, the image 812 may be segmented. Segmentation of the image 812 may be used to determine and/or delineate objects or portions in the image. The delineation may include or be made as a mask that is represented on a display. The representation may be shown on the display, such as with an icon. In some examples, the delineation may be used to identify boundaries of various portions within the image 812, such as boundaries of one or more structures of the patient 806 (such as spinal vertebrae 806v), among other examples. Accordingly, the image 812 may include an image of one or more of the spinal vertebrae 806v, such as a first vertebra 806vi and a second vertebra 806vii. The spinal vertebrae 806v may be delineated in the image 812, which may include and/or assist in determining boundaries in the image 812. In some examples, the delineation may be represented using a first icon 806vi′ or a second icon 806vii′. The first icon 806vi′ or the second icon 806vii′ may be used to represent, for display, a selected item, including the delineation of the object, and/or the boundary, among other examples.

As indicated above, FIG. 8 is provided as an example. Other examples may differ from what is described with regard to FIG. 8.

The adjustable arm assembly, including the base mount 102, the first segment 104, the second segment 106, and the display mount 108, may enable a user (e.g., the user 802) to move the display device (e.g., the display device 814) to various placements and/or orientations, such as during medical imaging procedures. Thus, the user may control medical imaging parameters and/or perform medical imaging operations from any suitable location (rather than only near the backside of the gantry). Additionally, or alternatively, the display device connected to the adjustable arm assembly may enable display of acquired medical images, thereby mitigating a need for a separate mobile view station and, thus, increasing available space in the OT.

The second segment being configured to rotate 360 degrees about the second vertical axis, the second segment being configured to rotate at least 120 degrees about the first lateral axis, and the display mount being configured to rotate at least 180 degrees about the second lateral axis, may enable the adjustable arm assembly to enter a collapsed state. For example, the collapsed state may prevent the adjustable arm assembly and/or the display device from obstructing the view of a person who is transporting (e.g., pushing) the medical imaging system 800.

The following provides an overview of some Aspects of the present disclosure:
Aspect 1: An adjustable arm assembly for a medical imaging system, comprising: a base mount; a first segment affixed to the base mount and configured to rotate about a first vertical axis; a second segment affixed to the first segment and configured to rotate about a second vertical axis and a first lateral axis; and a display mount affixed to the second segment and configured to rotate about a third vertical axis, a second lateral axis, and a longitudinal axis.
Aspect 2: The adjustable arm assembly of Aspect 1, wherein the first segment is configured to rotate 360 degrees about the first vertical axis.
Aspect 3: The adjustable arm assembly of any of Aspects 1-2, wherein the second segment is configured to rotate 360 degrees about the second vertical axis.
Aspect 4: The adjustable arm assembly of any of Aspects 1-3, wherein the second segment is configured to rotate at least 120 degrees about the first lateral axis.
Aspect 5: The adjustable arm assembly of any of Aspects 1-4, wherein the display mount is configured to rotate at least 180 degrees about the third vertical axis.
Aspect 6: The adjustable arm assembly of any of Aspects 1-5, wherein the display mount is configured to rotate at least 180 degrees about the second lateral axis.
Aspect 7: The adjustable arm assembly of any of Aspects 1-6, wherein the display mount is configured to rotate at least 180 degrees about the longitudinal axis.
Aspect 8: The adjustable arm assembly of any of Aspects 1-7, wherein the second segment is configured to rotate 360 degrees about the second vertical axis, the second segment is configured to rotate at least 120 degrees about the first lateral axis, and the display mount is configured to rotate at least 180 degrees about the second lateral axis.
Aspect 9: A medical imaging system, comprising: a medical imaging device; and an adjustable arm assembly that includes: a base mount affixed to the medical imaging device; a first segment affixed to the base mount and configured to rotate about a first vertical axis; a second segment affixed to the first segment and configured to rotate about a second vertical axis and a first lateral axis; and a display mount affixed to the second segment and configured to rotate about a third vertical axis, a second lateral axis, and a longitudinal axis.
Aspect 10: The medical imaging system of Aspect 9, wherein the first segment is configured to rotate 360 degrees about the first vertical axis.
Aspect 11: The medical imaging system of any of Aspects 9-10, wherein the second segment is configured to rotate 360 degrees about the second vertical axis.
Aspect 12: The medical imaging system of any of Aspects 9-11, wherein the second segment is configured to rotate at least 120 degrees about the first lateral axis.
Aspect 13: The medical imaging system of any of Aspects 9-12, wherein the display mount is configured to rotate at least 180 degrees about the third vertical axis.
Aspect 14: The medical imaging system of any of Aspects 9-13, wherein the display mount is configured to rotate at least 180 degrees about the second lateral axis.
Aspect 15: The medical imaging system of any of Aspects 9-14, wherein the display mount is configured to rotate at least 180 degrees about the longitudinal axis.
Aspect 16: The medical imaging system of any of Aspects 9-15, wherein the second segment is configured to rotate 360 degrees about the second vertical axis, the second segment is configured to rotate at least 120 degrees about the first lateral axis, and the display mount is configured to rotate at least 180 degrees about the second lateral axis.
Aspect 17: An adjustable arm assembly for a medical imaging system, comprising: a base mount; a first segment affixed to the base mount and configured to rotate about a first vertical axis; a second segment affixed to the first segment and configured to rotate 360 degrees about a second vertical axis and at least 120 degrees about a first lateral axis; and a display mount affixed to the second segment and configured to rotate about a third vertical axis, at least 180 degrees about a second lateral axis, and about a longitudinal axis.
Aspect 18: The adjustable arm assembly of Aspect 17, wherein the first segment is configured to rotate 360 degrees about the first vertical axis.
Aspect 19: The adjustable arm assembly of any of Aspects 17-18, wherein the display mount is configured to rotate at least 180 degrees about the third vertical axis.
Aspect 20: The adjustable arm assembly of any of Aspects 17-19, wherein the display mount is configured to rotate at least 180 degrees about the longitudinal axis.
Aspect 21: A system configured to perform one or more operations recited in one or more of Aspects 1-20.
Aspect 22: An apparatus comprising means for performing one or more operations recited in one or more of Aspects 1-20.

The foregoing disclosure provides illustration and description, but is not intended to be exhaustive or to limit the aspects described herein to the precise forms that are described. Modifications and variations may be made in light of the above description or may be acquired from practice of the aspects described herein.

As used herein, the term “component” is intended to be broadly construed as hardware, firmware, and/or a combination of hardware and software. It will be apparent that systems and/or methods described herein may be implemented in different forms of hardware, firmware, or a combination of hardware and software. The actual specialized control hardware or software code used to implement these systems and/or methods is not limiting of the aspects described herein. Thus, the operation and behavior of the systems and/or methods are described herein without reference to specific software code—it being understood that software and hardware can be designed to implement the systems and/or methods based on the description herein.

Even though particular combinations of features are recited in the claims and/or described in the specification, these combinations are not intended to limit the aspects described herein. In fact, many of these features may be combined in ways not specifically recited in the claims and/or described in the specification. Although each dependent claim listed below may directly depend on only one claim, the description includes each dependent claim in combination with every other claim in the claim set. As used herein, a phrase referring to “at least one of” a list of items refers to any combination of those items, including single members. As an example, “at least one of: a, b, or c” is intended to cover a, b, c, a-b, a-c, b-c, and a-b-c, as well as any combination with multiple of the same item.

When “a component” or “one or more components” (or another element, such as “a controller” or “one or more controllers”) is described or claimed (within a single claim or across multiple claims) as performing multiple operations or being configured to perform multiple operations, this language is intended to broadly cover a variety of architectures and environments. For example, unless explicitly claimed otherwise (e.g., via the use of “first component” and “second component” or other language that differentiates components in the claims), this language is intended to cover a single component performing or being configured to perform all of the operations, a group of components collectively performing or being configured to perform all of the operations, a first component performing or being configured to perform a first operation and a second component performing or being configured to perform a second operation, or any combination of components performing or being configured to perform the operations. For example, when a claim has the form “one or more components configured to: perform X; perform Y; and perform Z,” that claim should be interpreted to mean “one or more components configured to perform X; one or more (possibly different) components configured to perform Y; and one or more (also possibly different) components configured to perform Z.”

No element, act, or instruction used herein should be construed as critical or essential unless explicitly described as such. Also, as used herein, the articles “a” and “an” are intended to include one or more items, and may be used interchangeably with “one or more.” Further, as used herein, the article “the” is intended to include one or more items referenced in connection with the article “the” and may be used interchangeably with “the one or more.” Furthermore, as used herein, the term “set” is intended to include one or more items (e.g., related items, unrelated items, or a combination of related and unrelated items,), and may be used interchangeably with “one or more.” Where only one item is intended, the phrase “only one” or similar language is used. Also, as used herein, the terms “has,” “have,” “having,” or the like are intended to be open-ended terms. Further, the phrase “based on” is intended to mean “based, at least in part, on” unless explicitly stated otherwise. Also, as used herein, the term “or” is intended to be inclusive when used in a series and may be used interchangeably with “and/or,” unless explicitly stated otherwise (e.g., if used in combination with “either” or “only one of”). Further, spatially relative terms, such as “below,” “lower,” “above,” “upper,” and the like, may be used herein for ease of description to describe one element or feature's relationship to another element(s) or feature(s) as illustrated in the figures. The spatially relative terms are intended to encompass different orientations of the apparatus, device, and/or element in use or operation in addition to the orientation depicted in the figures. The apparatus may be otherwise oriented (rotated 90 degrees or at other orientations) and the spatially relative descriptors used herein may likewise be interpreted accordingly.

What is claimed is:

1. An adjustable arm assembly for a medical imaging system, comprising:
a base mount;
a first segment affixed to the base mount and configured to rotate about a first vertical axis;
a second segment affixed to the first segment and configured to rotate about a second vertical axis and a first lateral axis; and
a display mount affixed to the second segment and configured to rotate about a third vertical axis, a second lateral axis, and a longitudinal axis.

2. The adjustable arm assembly of claim 1, wherein the first segment is configured to rotate 360 degrees about the first vertical axis.

3. The adjustable arm assembly of claim 1, wherein the second segment is configured to rotate 360 degrees about the second vertical axis.

4. The adjustable arm assembly of claim 1, wherein the second segment is configured to rotate at least 120 degrees about the first lateral axis.

5. The adjustable arm assembly of claim 1, wherein the display mount is configured to rotate at least 180 degrees about the third vertical axis.

6. The adjustable arm assembly of claim 1, wherein the display mount is configured to rotate at least 180 degrees about the second lateral axis.

7. The adjustable arm assembly of claim 1, wherein the display mount is configured to rotate at least 180 degrees about the longitudinal axis.

8. The adjustable arm assembly of claim 1, wherein the second segment is configured to rotate 360 degrees about the second vertical axis, the second segment is configured to rotate at least 120 degrees about the first lateral axis, and the display mount is configured to rotate at least 180 degrees about the second lateral axis.

9. A medical imaging system, comprising:
a medical imaging device; and
an adjustable arm assembly that includes:
a base mount affixed to the medical imaging device;
a first segment affixed to the base mount and configured to rotate about a first vertical axis;
a second segment affixed to the first segment and configured to rotate about a second vertical axis and a first lateral axis; and
a display mount affixed to the second segment and configured to rotate about a third vertical axis, a second lateral axis, and a longitudinal axis.

10. The medical imaging system of claim 9, wherein the first segment is configured to rotate 360 degrees about the first vertical axis.

11. The medical imaging system of claim 9, wherein the second segment is configured to rotate 360 degrees about the second vertical axis.

12. The medical imaging system of claim 9, wherein the second segment is configured to rotate at least 120 degrees about the first lateral axis.

13. The medical imaging system of claim 9, wherein the display mount is configured to rotate at least 180 degrees about the third vertical axis.

14. The medical imaging system of claim 9, wherein the display mount is configured to rotate at least 180 degrees about the second lateral axis.

15. The medical imaging system of claim 9, wherein the display mount is configured to rotate at least 180 degrees about the longitudinal axis.

16. The medical imaging system of claim 9, wherein the second segment is configured to rotate 360 degrees about the second vertical axis, the second segment is configured to rotate at least 120 degrees about the first lateral axis, and the display mount is configured to rotate at least 180 degrees about the second lateral axis.

17. An adjustable arm assembly for a medical imaging system, comprising:
a base mount;
a first segment affixed to the base mount and configured to rotate about a first vertical axis;
a second segment affixed to the first segment and configured to rotate 360 degrees about a second vertical axis and at least 120 degrees about a first lateral axis; and
a display mount affixed to the second segment and configured to rotate about a third vertical axis, at least 180 degrees about a second lateral axis, and about a longitudinal axis.

18. The adjustable arm assembly of claim 17, wherein the first segment is configured to rotate 360 degrees about the first vertical axis.

19. The adjustable arm assembly of claim 17, wherein the display mount is configured to rotate at least 180 degrees about the third vertical axis.

20. The adjustable arm assembly of claim 17, wherein the display mount is configured to rotate at least 180 degrees about the longitudinal axis.
